(19) United States
(12) Patent Application Publication
(10) Pub. No.: US 2015/0276740 A1
(43) Pub. Date: Oct. 1, 2015

(54) METHOD FOR DETERMINING KINASE ACTIVITY

(71) Applicant: SYSMEX CORPORATION, Kobe-shi (JP)

(72) Inventor: Shigeyoshi HARADA, Kobe (JP)

(73) Assignee: SYSMEX CORPORATION, Kobe-shi (JP)

(21) Appl. No.: 14/632,517

(22) Filed: Feb. 26, 2015

(30) Foreign Application Priority Data
Mar. 31, 2014 (JP) 2014-072967

(51) Int. Cl. G01N 33/573

(52) U.S. Cl. CPC G01N 33/573; G01N 2333/912

(57) ABSTRACT

Disclosed is a method for determining kinase activity. The method comprises: (A) mixing a specimen that comprises a kinase, a substrate of the kinase and an adenosine triphosphate (ATP) derivative that comprises a dinitrophenyl (DNP) group to obtain a mixture that comprises a DNP group-containing substrate in which the DNP group is introduced in the substrate; (B) mixing the mixture obtained at the (A) and an antibody that binds to the DNP group to form a complex that comprises the DNP group-containing substrate and the antibody; and (C) determining an activity of the kinase by detecting the complex. The ATP derivative is a compound in which the DNP group is bound to a phosphate group at a gamma position of ATP via a linker.

### CROSS REFERENCE TO RELATED APPLICATIONS

This application claims priority from prior Japanese Patent Application No. 2014-072967, filed on Mar. 31, 2014, entitled “METHOD FOR DETERMINING KINASE ACTIVITY”, the entire content of which is incorporated herein by reference.

### TECHNICAL FIELD

The present invention relates to a method for determining kinase activity. In further detail, the present invention relates to an adenosine triphosphate derivative, a method for determining kinase activity using the same, and a reagent kit thereof.

### BACKGROUND

As a method for determining kinase activity, for example, instead of using adenosine triphosphate (hereinafter, referred to as “ATP”) as a phosphate-group donor, a method for determining the activity of cyclin-dependent kinase using adenosine 5′-O-(3-thiotriphosphate) (hereinafter, referred to as “ATPγS”) has been proposed (cf. specification of U.S. Patent Application Publication No. 20020164673).

The method disclosed in the specification of U.S. Patent Application Publication No. 20020164673 is performed in the following manner. First, in the presence of a cyclin-dependent kinase/cyclin complex, a reaction is caused to occur between a substrate protein and ATPγS to introduce an ATPγS derived monothiophosphate group to a serine residue or a threonine residue of the substrate protein. Next, a fluorescence labeled substance or a labeling enzyme is coupled to a sulfur atom of the introduced monothiophosphate group to obtain a labeled substrate protein. Then, an activity value of cyclin-dependent kinase is calculated based on a level of fluorescence derived from the fluorescence labeled substance of the labelled substrate protein, or an amount of product produced from a reaction by the labeling enzyme.

### SUMMARY

However, there has been a demand for a method capable of determining kinase activity with higher sensitivity.

Thus, the present invention provides a method for determining kinase activity, the method including:

(A) mixing a specimen that comprises a kinase, a substrate of the kinase and an adenosine triphosphate (ATP) derivative that comprises a dinitrophenyl (DNP) group to obtain a mixture that comprises a DNP group-containing substrate in which the DNP group is introduced in the substrate;

(B) mixing the mixture obtained at the (A) and an antibody that binds to the DNP group to form a complex that comprises the DNP group-containing substrate and the antibody; and

(C) determining an activity of the kinase by detecting the complex,

wherein the ATP derivative is a compound in which the DNP group is bound to a phosphate group at a gamma position of ATP via a linker.

### BRIEF DESCRIPTION OF THE DRAWINGS

FIG. 1 is an outline explanatory view showing the principle of a method for determining kinase activity according to an embodiment;

FIG. 2A shows an absorbance spectrum of ATPγS used in Example 1;

FIG. 2B shows an absorbance spectrum of DNP-Lys used in Example 1;

FIG. 2C shows an absorbance spectrum of a reaction product obtained in Example 1;

FIG. 3 is a graph showing a result of Example 1;

FIG. 4 is a graph showing a result of Example 1; and

FIG. 5 is a graph showing results evaluating both determining methods of Example 2 and Comparative Example 1.

### DETAILED DESCRIPTION OF THE PREFERRED EMBODIMENTS

(ATP Derivative)

A characteristic of an ATP derivative according to the present embodiment is that a DNP group is bound to a phosphate group at the gamma position of ATP via a linker. The ATP derivative according to the present embodiment is formed from an ATP portion and a DNP group portion.

As described herein, “kinase” refers to an enzyme that transfers a phosphate group from a compound such as ATP having a high-energy phosphate bond to a substrate to cause phosphorylation of the substrate. The kinase encompasses low-molecular-weight substrate type kinases that cause phosphorylation of a low molecular weight compound which is a substrate, protein kinases that cause phosphorylation of a protein, which is a substrate, having a specific amino acid sequence, and the like. Examples of the low-molecular-weight substrate type kinases include, but are not particularly limited to, creatine kinase, pyruvate kinase, and the like. The protein kinases are classified largely into serine/threonine kinases and tyrosine kinases. Examples of the serine/threonine kinases include, but are not particularly limited to: CDKs such as cyclin-dependent kinase (CDK) 1, CDK2, CDK4, and CDK6; Akt 1; IKK; PDK1; PK; PKA; PKC; PKN; Rsk1; Rsk2; SGK; KSR; LKB1; MAPK1; MAPK2; PAK3; PKR; PLK1; PRAK; PRK2; Raf; and Tak1. Examples of the tyrosine kinases include, but are not particularly limited to: receptor tyrosine kinases such as Eck, EGF-R, Erb B-2, Erb B-3, Erb B-4, FGF-R, Flt-1, PDGF-R, TrkA, TrkB, TrkC, and Tie-2; and non-receptor tyrosine kinases such as Abl, FAK, Pyk2, Yes, Csk, Fyn, Lck, Tec, Blk, JAK1, JAK2, JAK3, Src, Eck, Hck, Lyn, Tyk2, BTK, Fgr, and Syk. Among the kinase described above, a CDK is preferable, and CDK1 or CDK2 is more preferable.

Examples of the ATP derivative according to present embodiment include, but are not particularly limited to, a compound represented by formula (I):

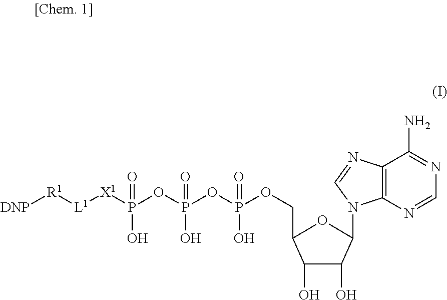

(wherein, X¹ represents a direct binding, an oxygen atom, or a sulfur atom, L¹ represents a linker portion, R¹ represents a reactive group that can be coupled to both the L¹ and the DNP, and DNP represents a dinitrophenyl group). In the present embodiment, from a standpoint of ensuring ease of producing the ATP derivative, R¹ and X¹ are preferably functional groups that are different from each other.

In formula (I), X¹ is a direct binding, an oxygen atom, or a sulfur atom. Among the X¹ described above, a sulfur atom is preferable from a standpoint of ensuring ease of producing the ATP derivative.

In formula (I), L¹ is a linker portion. The linker portion is a portion that is produced by coupling, with respect to R¹ and X¹, a bifunctional linker having a first reactive group that can be coupled to R¹ and a second reactive group that can be coupled to X¹. Thus, a functional group at a terminal on a side of R¹ in L¹ is a functional group (hereinafter, referred to as “R¹ binding group”) derived from the first reactive group. Furthermore, a functional group at a terminal on a side of X¹ in L¹ is a functional group (hereinafter, referred to as “X¹ binding group”) derived from the second reactive group.

The R¹ binding group can be selected as appropriate depending on the type of R¹. When R¹ is an amino acid residue, examples of the R¹ binding group include, but are not particularly limited to, carbonyl group, amino group, and sulfhydryl group. The R² binding group can be selected as appropriate depending on the type of X². When X¹ is a sulfur atom, examples of the X¹ binding group include, but are not particularly limited to, maleimide group, bromoacetamide group, iodoacetamide group, and disulfide group. If necessary, the linker portion may have a spacer interposed between the R¹ binding group and the X¹ binding group. Examples of the spacer include, but are not particularly limited to, an alkylene group having a carbon number of 1 to 12 and optionally having a substituent group, an alkenylene group having a carbon number of 2 to 12 and optionally having a substituent group, an alkynylene group having a carbon number of 2 to 12 and optionally having a substituent group, and a (poly)oxyalkylene group. The carbon number of the alkylene group is: from a standpoint of enabling ATP to sufficiently function through suppression of steric hindrance between the ATP portion and the DNP portion of the ATP derivative and efficiently performing phosphorylation, preferably not smaller than 1 and more preferably not smaller than 2; and, from a standpoint of ensuring ease of operation by ensuring water solubility of the ATP derivative, preferably not larger than 12, more preferably not larger than 8, and further preferably not larger than 6. Examples of the alkylene group whose carbon number is 1 to 12 include, but are not particularly limited to, methylene group, ethylene group, n-propylene group, isopropylene group, n-butylene group, isobutylene group, sec-butylene group, tert-butylene group, n-pentylene group, isopentylene group, neopentylene group, and hexylene group. Each of the carbon numbers of the alkenylene group and the alkynylene group is: from a standpoint of enabling ATP to sufficiently function through suppression of steric hindrance between the ATP portion and the DNP portion of the ATP derivative and efficiently performing phosphorylation, preferably not smaller than 2 and more preferably not smaller than 3; and, from a standpoint of ensuring ease of operation by ensuring water solubility of the ATP derivative, preferably not larger than 12, more preferably not larger than 8, and further preferably not larger than 6. Examples of the alkenylene group whose carbon number is 2 to 12 include, but are not particularly limited to, vinylene group, propenylene group, butenylene group, and pentenylene group. Examples of the alkynylene group whose carbon number is 2 to 12 include, but are not particularly limited to, ethynylene group, propynylene group, butynylene group, and hexenylene group. Examples of the substituent group include, but are not particularly limited to, hydroxyl group and amino group. Examples of the (poly)oxyalkylene group include, but are not particularly limited to, a (poly)oxyalkylene group in which the carbon number of an oxyalkylene group is 1 to 12 and the number of added moles of the oxyalkylene group is 1 to 8. The carbon number of the oxyalkylene group is: from a standpoint of enabling ATP to sufficiently function through suppression of steric hindrance between the ATP portion and the DNP portion of the ATP derivative and efficiently performing phosphorylation, preferably not smaller than 1 and more preferably not smaller than 2; and, from a standpoint of ensuring ease of operation by ensuring water solubility of the ATP derivative, preferably not larger than 12, more preferably not larger than 8, and further preferably not larger than 6. Examples of the oxyalkylene group whose carbon number is 1 to 12 include, but are not particularly limited to, oxymethylene group, oxyethylene group, oxypropylene group, and oxybutylene group.

In formula (I), R¹ is a reactive group that can be coupled to both L¹ and DNP. Examples of the reactive group that can be coupled to both L¹ and DNP include, but are not particularly limited to, an amino acid residue and an aminoalkyl carboxylic acid in which the carbon number of an alkyl group is 1 to 4. Examples of the amino acid residue include, but are not particularly limited to, alanine residue, glycine residue, leucine residue, lysine residue, methionine residue, phenylalanine residue, proline residue, serine residue, threonine residue, and valine residue. It should be noted that the amino acid residue at R¹ refers to a divalent group derived from an amino acid. Among the R¹ described above, lysine residue is preferable from a standpoint of ensuring reactivity of the reactive group and ease of production.

(Method for Producing ATP Derivative)

For example, the ATP derivative according to the present embodiment can be produced by causing a reaction to occur among a DNP group-containing compound represented by formula (II):

DNP-R² (II)

(wherein, R² represents an amino acid residue, and DNP is identical to that described above), an ATP compound represented by formula (III):

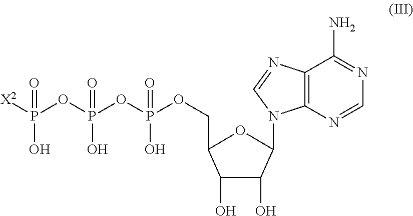

(wherein, X² represents a reactive group (excluding a functional group-containing DNP)), and

a bifunctional linker having the first reactive group that can be coupled to R² and the second reactive group that can be coupled to X². In the present embodiment, the order in which the DNP group-containing compound represented by formula (II), the ATP compound represented by formula (III), and the bifunctional linker are caused to react is not particularly limited. The reaction can be caused to occur among the DNP group-containing compound represented by formula (II), the ATP compound represented by formula (III), and the bifunctional linker by, for example,
(1) causing a reaction to occur between the DNP group-containing compound represented by formula (II) and the bifunctional linker, and then causing a reaction to occur between the obtained production intermediate and the ATP compound represented by formula (III), or (2) causing a reaction to occur between the ATP compound represented by formula (III) and the bifunctional linker, and then causing a reaction to occur between the obtained production intermediate and the DNP group-containing compound represented by formula (II).

In the following, although description will be provided using the production method of (1) described above as an example, the production method is not limited thereto. First, a reaction is caused to occur between the DNP group-containing compound represented by formula (II) and the bifunctional linker to obtain a production intermediate represented by formula (IV):

DNP-R¹-L² (IV)

(wherein, DNP and R¹ are each identical to those described above, and L² is a linker portion derived from the bifunctional linker) (step 1-1). The reaction at step 1-1 can be performed in, for example, an organic solvent such as N,N-dimethylformamide. When performing the reaction at step 1-1, any reaction temperature may be used as long as the temperature is sufficient for causing a reaction to occur between the DNP group-containing compound and the bifunctional linker. Although the reaction temperature is not particularly limited, the temperature is ordinarily 15 to 40° C., and preferably 25 to 35° C. When performing the reaction at step 1-1, any reaction time may be used as long as the time is sufficient for causing a reaction to occur between the DNP group-containing compound and the bifunctional linker. The reaction time is ordinarily 0.5 to 3 hours, and preferably 0.5 to 2 hours. A reaction product obtained from the reaction includes the production intermediate of the ATP derivative. The reaction product can be further concentrated if necessary by appropriately purifying the reaction product using reversed-phase high performance liquid chromatography or the like. At step 1-1, the amount of the bifunctional linker per 1 mol of the DNP group-containing compound represented by formula (II) is: from a standpoint of improving yield of the production intermediate, preferably not less than 0.5 mol and more preferably not less than 0.6 mol; and, from a standpoint of suppressing side reactions at the next step, preferably not more than 2 mol, more preferably not more than 1 mol, and further preferably not more than 0.8 mol.

In formula (II), R² is an amino acid residue. Examples of the amino acid residue include, but are not particularly limited to, alanine residue, glycine residue, leucine residue, lysine residue, methionine residue, phenylalanine residue, proline residue, serine residue, threonine residue, and valine residue. It should be noted that the amino acid residue at R² refers to a monovalent group derived from an amino acid. Among the R² described above, lysine residue is preferable from a standpoint of ensuring reactivity of the reactive group and ease of production.

The bifunctional linker consists of a bifunctional linker having a first reactive group that can be coupled to R² and a second reactive group that can be coupled to X². The first reactive group can be selected as appropriate depending on the type of R². When R² is an amino acid residue, and the DNP group-containing compound represented by formula (II) and the bifunctional linker are to be coupled via the amino group of the amino acid residue, examples of the first reactive group include, but are not particularly limited to, carbonyl group, isothiocyano group, chlorosulfone group, chlorocarbonyl group, carboxyl group, and succinimide group. The second reactive group can be selected as appropriate depending on the type of X². For example, when X² is a sulfur atom, examples of the second reactive group include, but are not particularly limited to, maleimide group, bromoacetamide group, iodoacetamide group, and disulfide group. The linker portion may have a spacer interposed between the first reactive group and the second reactive group if necessary. Examples of the spacer include spacers similar those described above. Examples of the bifunctional linker include, but are not particularly limited to, (maleimidoalkynoyloxy)succinimides in which the carbon number of alkynoyl group is 1 to 12, such as N-(4-maleimidobutyryloxy)succinimide, N-(6-maleimidohexanoyloxy)succinimide, N-(8-maleimidooctanoyloxy)succinimide, and N-(11-maleimidoundecanoyloxy)succinimide.

In formula (IV), L² is a linker portion derived from the bifunctional linker. L² has a second reactive group that is free and that can be coupled to X¹.

Next, a reaction is caused to occur between the production intermediate obtained at step 1-1 and the ATP compound represented by formula (III) to obtain the ATP derivative according to the present embodiment (step 1-2). For example, the reaction at step 1-2 can be performed in a neutral water-based solvent such as sodium phosphate buffer. When performing the reaction at step 1-2, any reaction temperature may be used as long as the temperature is proper for causing a reaction to occur between the production intermediate represented by formula (IV) and the ATP compound represented by formula (III). Although the reaction temperature is not particularly limited, the temperature is ordinarily 15 to 40° C., and preferably 25 to 35° C. When performing the reaction at step 1-2, any reaction time may be used as long as the time is sufficient for causing a reaction to occur between the production intermediate represented by formula (IV) and the ATP compound represented by formula (III). The reaction time is ordinarily 0.5 to 3 hours, and preferably 0.5 to 2 hours. For example, the reaction can be terminated by adding a reaction terminator such as mercaptoethylamine to a reaction system. A reaction product including the obtained production intermediate can be further concentrated if necessary by appropriately purifying the reaction product using reversed-phase high performance liquid chromatography or the like. At step 1-2, the amount of the ATP compound represented by formula (III) per 1 mol of the production intermediate represented by formula (IV) is: from a standpoint of improving yield, preferably not less than 0.5 mol, more preferably not less than 0.6 mol, and further preferably not less than 0.8 mol; and, from a standpoint of reducing production cost and ease of purification at the next step, preferably not more than 2 mol, more preferably not more than 1.2 mol, and further preferably not more than 1.0 mol.

In formula (III), X² is a reactive group, but excluding the case where X² is a functional group including DNP in formula (III). Examples of the reactive group include, but are not particularly limited to, sulfhydryl group (—SH). From a standpoint of ensuring ease of producing the ATP derivative, X² is preferably a functional group that is different from R² in formula (II). For example, when R² is an amino acid residue, X² is preferably a functional group other than the amino acid residue. Among the X² described above, sulfhydryl group is preferable from a standpoint of ensuring ease of producing the ATP derivative.

For example, the obtained ATP derivative can be preserved after being dissolved in a solvent suitable for the use application.

The ATP derivative according to the present embodiment can be used as a phosphate-group donor in phosphorylation by a kinase. Thus, the ATP derivative can be suitably used in a later described method for determining kinase activity.

(Method for Determining Kinase Activity)

The method for determining kinase activity (hereinafter, referred to as “determining method of the present embodiment”), according to the present embodiment, includes the steps of:

(A) causing a reaction to occur among a specimen that contains a kinase, a substrate of the kinase, and the above described ATP derivative to obtain a mixture that contains a DNP group-containing substrate in which a DNP group is introduced in the substrate;

(B) mixing the mixture obtained at step (A) and an antibody that binds to the DNP group to form a complex that includes the DNP group-containing substrate and the antibody; and

(C) determining an activity of the kinase by detecting the complex, wherein the ATP derivative is a compound in which the dinitrophenyl group is bound to a phosphate group at the gamma position of the ATP via a linker.

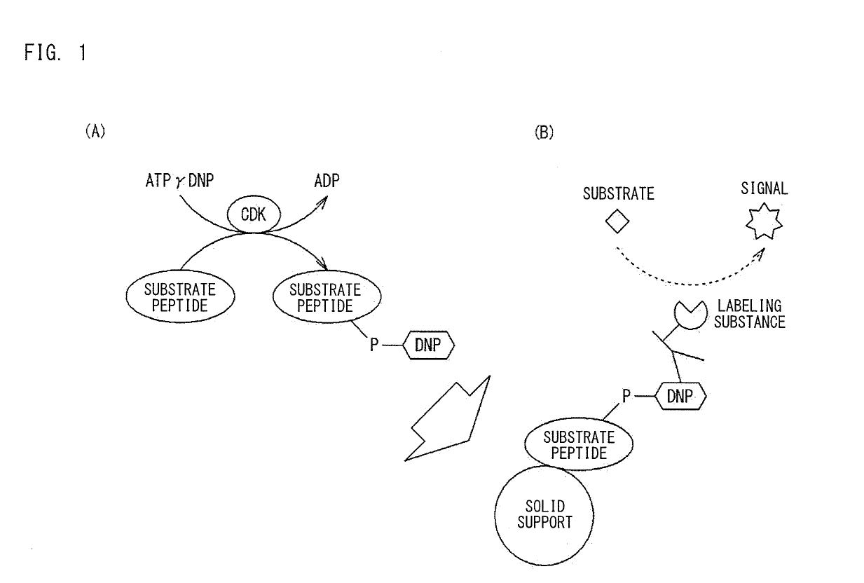

The principle of the determining method of the present embodiment is shown in FIG. 1. In FIG. 1, description is provided using CDK1, which is a kinase, as an example. In the figure, “ATPγDNP” represents the ATP derivative described above, “CDK” represents CDK1, “substrate peptide” represents a peptide having a motif sequence of a phosphorylation site for the CDK1, “labeling substance” represents an enzyme, “substrate” represents a substrate of the enzyme, and “signal” represents a signal generated when the enzyme acts on the substrate. As shown in (A) of FIG. 1, the ATP derivative (ATPγDNP) is used as the phosphate-group donor for the reaction by CDK1. By the action of CDK1, the phosphate group including the DNP group is transferred from the ATP derivative (ATPγDNP) to a serine residue or a threonine residue of the substrate peptide. Then, as shown in (B) of FIG. 1, the substrate peptide including the DNP group is immobilized on a solid support, unreacted ATPγDNP is separated therefrom, the DNP group of the substrate peptide including the DNP group is captured by an anti-DNP antibody, and the signal generated when the enzyme, which is the labeling substance, acts on the substrate is detected.

In the following, the procedure of the determining method of the present embodiment will be described in detail. In the determining method of the present embodiment, first, a reaction is caused to occur among a specimen containing a kinase, a substrate of the kinase, and the ATP derivative to obtain a mixture containing a DNP group-containing substrate in which a DNP group is introduced in the substrate (step (A)). At the present step (A), when the kinase in the specimen, the substrate of the kinase, and the ATP derivative are brought in contact, the order in which the contact occurs is not particularly limited.

The kinase that becomes a measuring target in the determining method of the present embodiment is a kinase described above. As the specimen containing the kinase, an organism-derived sample obtained from cells and body fluid of an organism can be used. Examples of the organism-derived sample include cells from stomach, liver, breast, mammary glands, lungs, pancreas, pancreatic glands, uterus, skin, esophagus, larynx, pharynx, tongue, and thyroid glands, and body fluid such as blood, urine, and lymph fluid.

Among kinases, as in the case with a CDK, there are enzymes that are activated when binding to, in the cytoplasm, a cyclin, which is a component existing in a cell nucleus, to enter a state (activated state) in which an enzyme activity is expressed. Furthermore, some types of kinases exist inward of a cell membrane or inside a cell nucleus, and are not exposed on the surface of the cell. When the kinase is an enzyme that is activated when binding to an intracellular component or a kinase that exists inward of a cell membrane or inside a cell nucleus, the specimen containing the kinase is preferably a solubilized sample obtained by destroying the cell membrane or nuclear membrane of the cell to release the kinase that is the measuring target.

The solubilized sample is obtained by performing solubilization treatment against cells of an organism. The solubilization treatment can be performed by subjecting cells of the biological specimen to ultrasonication or agitation through aspiration by a pipette in a buffer for solubilization treatment (hereinafter, referred to as “solubilizing agent”).

The solubilizing agent is a buffer containing a substance for destroying the cell membrane or the nuclear membrane. The solubilizing agent may further contain a substance for inhibiting denaturing or degradation of the kinase, a substance for suppressing degradation of a substrate that has been phosphorylated by the kinase, and the like.

Examples of the substance for destroying the cell membrane or the nuclear membrane include, but are not particularly limited to, surfactants and chaotropic agents. The surfactants can be used as long as the activity of the kinase which is the measuring target is not inhibited. Examples of the surfactants include polyoxyethylene alkyl phenyl ethers such as Nonidet P-40 (NP-40) and Triton X-100 (Registered trademark of Dow Chemical Company), deoxycholic acid, and CHAPS. With regard to the substance for destroying the cell membrane or the nuclear membrane, a single type may be used by itself, or a combination of two or more types may be used. The concentration of the substance for destroying the cell membrane or the nuclear membrane in the solubilizing agent is ordinarily 0.1 to 2 w/v %.

Examples of the substance for inhibiting denaturing or degradation of the kinase include, but are not particularly limited to, protease inhibitors. Examples of the protease inhibitors include, but are not particularly limited to, metalloprotease inhibitors such as EDTA and EGTA, serine protease inhibitors such as PMSF, trypsin inhibitors, and chymotrypsin, and cysteine protease inhibitors such as iodoacetamide and E-64. With regard to the substance for inhibiting denaturing or degradation of the kinase, a single type may be used by itself, or a combination of two or more types may be used. The concentration of the substance for inhibiting denaturing or degradation of the kinase in the solubilizing agent is ordinarily 0.5 to 10 mM in cases with EDTA, EGTA, and PMSF.

Examples of the substance for suppressing degradation of a substrate that has been phosphorylated by the kinase include, but are not particularly limited to, phosphatase inhibitors. Examples of the phosphatase inhibitors include, but are not particularly limited to, protein serine/threonine phosphatase inhibitors such as sodium fluoride, and protein tyrosine phosphatase inhibitors such as sodium orthovanadate. With regard to the substance for suppressing degradation of a substrate that has been phosphorylated by the kinase, a single type may be used by itself, or a combination of two or more types may be used. The concentration of the substance for suppressing degradation of a substrate that has been phosphorylated by the kinase in the solubilizing agent is ordinarily 25 to 250 mM in cases with sodium fluoride and 0.1 to 1 mM in cases with sodium orthovanadate.

The phosphorylation of the substrate by the kinase is performed in a reaction solution suitable for expressing the activity of the kinase. The reaction solution contains a buffer having a pH suitable for expressing the activity of the kinase, the substrate of the kinase, and the ATP derivative. If necessary, the reaction solution contains a metal cation required for expressing the activity of the kinase, such as magnesium ion and manganese ion. Examples of the buffer include a tris hydrochloride buffer and a HEPES buffer. The pH of the buffer can be determined as appropriate in accordance with the type of the kinase. For example, when the kinase is a CDK, the pH is ordinarily 6 to 8 and preferably 6.5 to 7.5.

The substrate of the kinase can be selected as appropriate depending on the type of the kinase. When the kinase is a low-molecular-weight substrate type kinase, for example, a low-molecular-weight substrate of the kinase such as creatine and pyruvate can be used as the substrate, but the substrate is not particularly limited thereto. When the kinase is a protein kinase, a substrate protein in accordance with the type of the protein kinase, and a substrate peptide having a motif sequence of a phosphorylation site for the protein kinase can be used as the substrate, but the substrate is not particularly limited thereto. The amount of the substrate of the kinase can be set as appropriate depending on the type of the kinase and the amount of the kinase. When the later described step (B) is to be performed on a solid support, the substrate may contain a substance that causes specific binding with high affinity such as biotin and streptavidin if necessary.

In the determining method of the present embodiment, the kinase is preferably a CDK. In this case, a substrate of the CDK which is the kinase can be used. The CDK is a positive cell-cycle regulator. In a cell, the CDK normally exists in the cytoplasm in an inactive form by itself, and the CDK itself becomes activated through phosphorylation to move into the nucleus from the cytoplasm. In the nucleus, the CDK binds to a cyclin that exists in the nucleus to form a complex of the CDK and cyclin, and forms an active form CDK after, if necessary, being subjected to further dephosphorylation. The active form CDK positively regulates the progression of the cell cycle at various stages of the cell cycle. The expression profile of the CDK and cyclin is considered relevant to specific cancers. Thus, determining the activity of the CDK using the determining method of the present embodiment has expectation of being able to acquire information regarding specific cancers. Examples of the CDK include, but are not particularly limited to, CDK1, CDK2, CDK3, CDK4, CDK5, CDK6, CDK7, and CDK8. When the kinase is CDK1 or CDK2, it is possible to use, as the substrate of the kinase, histone H1 and a substrate peptide having an amino acid sequence (SEQ ID NO: 1) represented by formula (a1):

Xaa¹-Pro-Xaa²-Xaa³ (a1)

(Xaa¹ represents a serine residue or a threonine residue, Xaa² represents any amino acid residue, and Xaa³ represents a lysine residue or an arginine residue).

The substrate peptide having the amino acid sequence represented by formula (a1) may be a peptide produced through chemical or genetic engineering, or a peptide that exists in nature.

The amount of the ATP derivative can be set as appropriate depending on the type of the kinase, and the amount of the kinase, etc.

The reaction temperature used when phosphorylation of the substrate is performed by the kinase can be set as appropriate depending on the type the kinase. The reaction temperature is ordinarily 30 to 37° C. The reaction time can be set as appropriate depending on the type of the kinase, and the amount of the kinase, etc. The reaction time is ordinarily 10 to 60 minutes.

The DNP group-containing substrate obtained at step (A) is a compound in which a DNP group-containing monophosphate in the ATP derivative is introduced to a phosphorylation site of the substrate. Thus, the activity of the kinase can be determined by determining the amount of the DNP group-containing substrate.

Next, in the determining method according to the present embodiment, the mixture obtained at step (A) and an antibody that binds to the DNP group are mixed to form a complex that includes the DNP group-containing substrate and the antibody (step (B)).

The antibody that binds to the DNP group is preferably a labeled antibody including a labeling substance, since detecting the complex becomes easy. The antibody that binds to the DNP group can be created easily by, for example, immunizing an animal using a compound containing the DNP group with a commonly used method such as a method described in Current Protocols in Immunology (edited by John E. Coligan (John Wiely & Sons, Inc.), published in 1992). Furthermore, labeling of the antibody using the labeling substance can be performed easily with a technique depending on the type of the labeling substance. In the present embodiment, instead of the antibody, an antibody fragment obtained by purifying the antibody and treating the antibody with peptidase, etc., may be used.

The labeling substance may be an enzyme or a fluorescent substance.

Examples of the fluorescent substance include, but are not particularly limited to: fluorescein derivatives such as iodoacetyl-fluorescein isothiocyanate, 5-(bromomethyl)fluorescein, fluorescein-5-maleimide, 5-iodoacetamide fluorescein, and 6-iodoacetamide fluorescein; coumarin derivatives such as 4-bromomethyl-7-methoxycoumarin; eosine derivatives such as eosine-5-maleimide and eosine-5-iodoacetamide; phenanthroline derivatives such as N-(1,10-phenanthroline-5-yl)bromoacetamide; pyrene derivatives such as 1-pyrenebutyryl chloride, N-(1-pyreneethyl)iodoacetamide, N-(1-pyrenemethyl)iodoacetamide, and 1-pyrenemethyl iodoacetate; and rhodamine derivatives such as Rhodamine Red C2 maleimide. Examples of the enzyme include, but are not particularly limited to, β-galactosidase, alkaline phosphatase, glucose oxidase, and peroxidase.

The complex that includes the DNP group-containing substrate and the antibody is preferably formed on a solid support. This is for enabling, at a later step, recovery of the complex with a simple operation, and efficient detection of the complex. Examples of the solid support include, but are not particularly limited to, magnetic beads, and microplates. When magnetic beads are to be used as the solid support, for example, streptavidin immobilized magnetic beads and biotin immobilized magnetic beads can be used as the magnetic beads. When using the streptavidin immobilized beads or the biotin immobilized magnetic beads, as the DNP group-containing substrate, a substrate including a substance that binds to a substance immobilized on the magnetic beads is used. For example, when using the streptavidin immobilized beads, the complex can be formed on the magnetic beads by using a biotinylated DNP group-containing substrate as the DNP group-containing substrate. Furthermore, when using the biotin immobilized beads, the complex can be formed on the magnetic bead by using a streptavidin labeled DNP group-containing substrate as the DNP group-containing substrate.

The formation of the complex can be performed in a solution. Any solution may be used as long as the solution is suitable for forming the complex. The solution contains: a buffer such as a tris hydrochloride buffer and a HEPES buffer; a salt such as sodium chloride; and a blocking agent such as bovine serum albumin (BSA); etc. The pH of the solution may be in a range that maintains the functions of the DNP group-containing substrate and the antibody. The pH of the solution is ordinarily 6 to 8 and preferably 6.5 to 7.5.

When performing the formation of the complex in the solution, a step of separating the solution and the solid support having the complex formed thereon may be performed between step (B) and a later described step (C). With this, contamination of nonspecific impurities and the like can be suppressed, and the accuracy in detecting the complex can be improved. The separating of the solution and the solid support can be performed by, for example, when the magnetic beads are used as the solid support, collecting the magnetic beads using a magnet to separate the solution and the solid support having the complex formed thereon. The separating of the solution and the solid support may be performed using centrifugation. Furthermore, from a standpoint of suppressing contamination of nonspecific impurities and the like and improving the accuracy in detecting the complex, if necessary, the solid support having formed thereon the complex may be cleaned using a cleaning liquid for cleaning solid supports.

Next, the activity of the kinase is determined by detecting the complex obtained at step (B) (step (C)).

With regard to the detection of the complex, when the antibody is a labeled antibody including a labeling substance, the activity can be determined by detecting the labeling substance in the complex. Specifically, when the labeling substance is a fluorescent substance, by irradiating the fluorescent substance with an excitation light in accordance with the fluorescent substance, fluorescent light is generated as a signal. By determining the amount (intensity) of the fluorescent light, the amount of the DNP group-containing substrate having the antibody bound thereto (reaction product) can be determined. In this case, the amount of the DNP group-containing substrate is calculated based on a determined value of the amount (intensity) of the fluorescent light, using a calibration curve created from a known amount of the DNP group-containing substrate and the amount (intensity) of fluorescent light. With this, the amount of the calculated DNP group-containing substrate can be obtained as an activity value of the kinase contained in the specimen.

Furthermore, when the labeling substance is an enzyme, luminescence is generated by causing the enzyme to act on an enzyme substrate that generates luminescence through a reaction with the enzyme. By detecting the amount (intensity) of the luminescence, the amount of the reaction product (DNP group-containing substrate) bound to the antibody can be determined. In this case, the amount of the DNP group-containing substrate is calculated based on a determined value of the amount (intensity) of the luminescence, using a calibration curve created from a known amount of the DNP group-containing substrate and the amount (intensity) of luminescence. With this, the amount of the calculated DNP group-containing substrate can be obtained as an activity value of the kinase contained in the specimen.

(Reagent Kit)

A reagent kit according to the present embodiment is a reagent kit to be used for the above described method for determining kinase activity, and includes a substrate of a kinase, the above described ATP derivative, and an antibody that binds to the DNP group in the ATP derivative. The substrate of the kinase and the antibody in the reagent kit according to the present embodiment are the same as the substrate of the kinase and the antibody used in the above described method for determining kinase activity. The substrate of the kinase may be immobilized on a solid support.

The reagent kit according to the present embodiment may be a reagent kit in which the substrate of the kinase, the ATP derivative, and the antibody are housed in separate containers, or maybe a reagent kit in which the substrate of the kinase, the ATP derivative, and the antibody are housed in the same container. In addition, the reagent kit of the present embodiment may further include substances necessary at the time of phosphorylation such as, for example, a solution containing a metal cation, depending on the type of the kinase. In addition, when the antibody is a labeled antibody having an enzyme as a labeling substance, the reagent kit may further include an enzyme substrate for the enzyme and a reaction solution for the enzyme.

### EXAMPLES

In the following, detailed description will be provided using Examples. In the following, “ATPγDNP” represents the ATP derivative containing the dinitrophenyl group according to the present embodiment, “ATPγS” represents adenosine 5′-(γ-thiotriphosphate)(5′-O-(dihydroxy thio phosphinyloxy phosphonyloxy phosphonyl)adenosine manufactured by Merck & Co., Inc.), “EMCS” represents N-(6-maleimidocaproyloxy)succinimide (manufactured by Dojindo Laboratories (Co., Ltd.)), “DNP-Lys” represents N-(2,4-dinitrophenyl)-L-lysine (manufactured by Tokyo Kasei Kogyo (Co., Ltd.), “DMF” represents N,N-dimethylformamide, and “SM(PEG)₂” represents succinimidyl-([N-maleimidopropionamide]-diethylene glycol)ester manufactured by Thermo Scientific Inc.).

### Example 1

### (1) Synthesis of ATPγDNP

96 mg of EMCS was dissolved in 3.2 mL of DMF, and then mixed with a 50 v/v % DMF solution containing 1.5 equivalent of DNP-Lys to obtain a 6 mL mixture. The obtained 6 mL mixture was diluted by 2.5-fold using 0.1M sodium phosphate (pH7.0). The obtained dilution was left still for 1 hour at 30° C. for causing a reaction to occur between the EMCS and the DNP-Lys. As a result, 15 mL of a solution containing a production intermediate (compound 1) represented by formula (x1) was obtained.

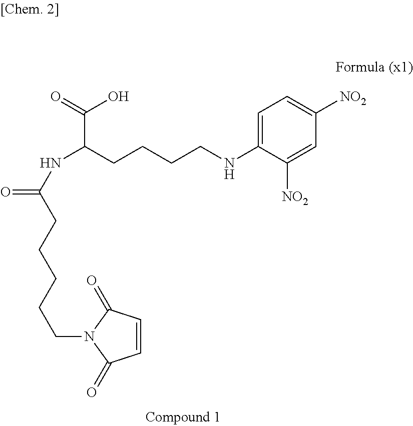 [SMILES: O=C(CCCCCN1C(=O)C=CC1=O)NC(CCCCNc1ccc([N+](=O)[O-])cc1[N+](=O)[O-])C(=O)O]

ATPγS was dissolved in 0.7 mL of ultrapure water by a mole number equal to that of EMCS used for the reaction with the DNP-Lys, and 14.5 mL of the solution containing the production intermediate (compound 1) was added thereto. Then, the obtained mixture was left still for 1 hour at 30° C. for causing a reaction to occur between the production intermediate (compound 1) and ATPγS. With respect to 15.2 mL of the obtained solution, a 1M mercaptoethylamine solution by an amount 1/20 of the volume of the solution was added, and the obtained mixture was left still for 5 minutes at 30° C. to terminate the reaction.

The solution containing the obtained reaction product was purified through reversed-phase chromatography. The purification conditions were those described in the following. Obtained fractions were concentrated through centrifugation to obtain a reaction product.

### <Purification Condition>

Detection wavelength: 260 nm and 360 nm

Used column: C18 reversed-phase column (product name: Redisep Rf C18 manufactured by Teledyne Isco Inc.)

Column temperature: Room temperature

Mobile phase

Mobile phase A: 50 mM tetraethylammonium bromide-containing aqueous solution

Mobile phase B: 50 mM tetraethylammonium bromide-containing acetonitrile solution
Concentration gradient for using mobile phases A and B
(Acetonitrile concentration: a concentration gradient of 0 to 40 v/v %)

Flow rate: 5 mL/min

### (2) Identification of Reaction Product

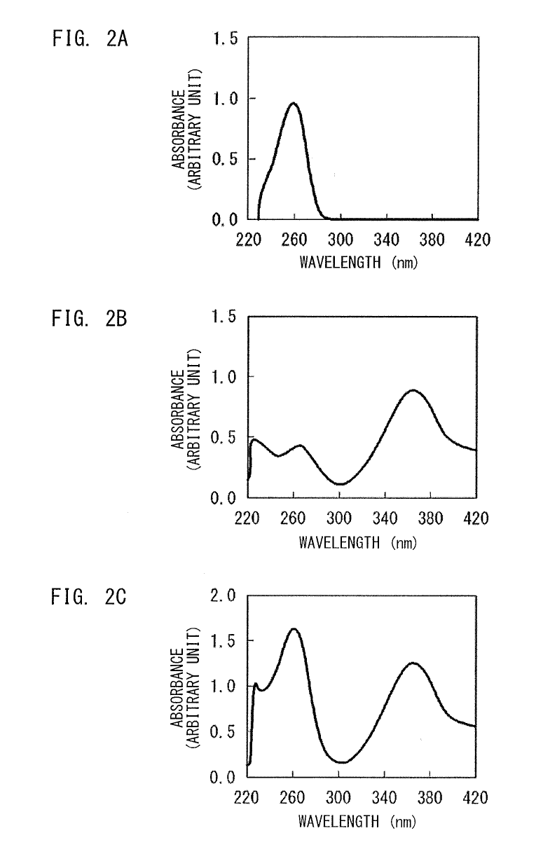

The obtained reaction product was diluted by 100-fold in a 50 mM tetraethylammonium bromide aqueous solution to obtain a measurement sample. By using the obtained measurement sample, a reference solution (50 mM tetraethylammonium bromide aqueous solution), and a spectrophotometer (product name: UV-1800PC manufactured by Shimadzu Corp.), an absorbance spectrum of wavelengths of 220 to 420 nm was measured. Furthermore, the absorbance spectrum was measured through a similar operation performed above, except for using ATPγS or DNP-Lys as the material instead of the reaction product. The absorbance spectrum of ATPγS, the absorbance spectrum of DNP-Lys, and the absorbance spectrum of the reaction product are respectively shown in FIG. 2A, FIG. 2B, and FIG. 2C.

In (1) described above, it is thought that a crosslink is formed between a maleimide group of the production intermediate (compound 1) and a thiol group of ATPγS when a reaction occurs between the production intermediate (compound 1) and the ATPγS. From the result shown in FIG. 2, the absorbance spectrum of the reaction product can be observed to have a characteristic peak of ATPγS at a wavelength of around 260 nm, and a characteristic peak of DNP-Lys at a wavelength of around 360 nm. Thus, the reaction product is suggested to be a compound 2 represented by formula (x2):

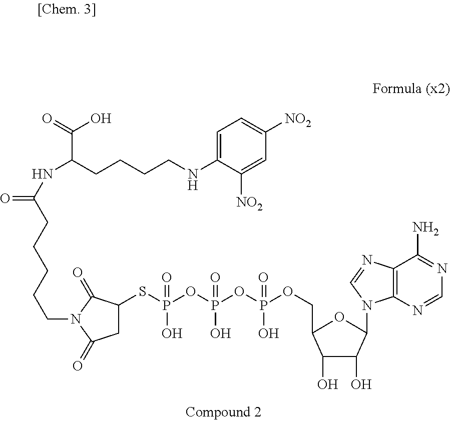 [SMILES: Nc1ncnc2c1ncn2C1OC(COP(=O)(O)OP(=O)(O)OP(=O)(O)SC2CC(=O)N(CCCCCC(=O)NC(CCCCNc3ccc([N+](=O)[O-])cc3[N+](=O)[O-])C(=O)O)C2=O)C(O)C1O]

The compound 2 represented by formula (x2) is one type (ATPγDNP) of the ATP derivative according to the present embodiment.

(2) Kinase Reaction (Transphosphorylation)

In a well of a 96 well filter plate (hydrophilic PVDF membrane manufactured by Millipore Corp.), 70 μL of an immunoprecipitation buffer (a buffer containing 0.1 mass % Nonidet NP-40 and 50 mM tris hydrochloride (pH7.4)) was added. Then, with respect to the immunoprecipitation buffer in the well, 20 μL of an antibody solution containing 16 μg of an anti-CDK1 antibody (manufactured by Operon Co., ltd.) or 8 μg of an anti-CDK2 antibody (manufactured by Operon Co., ltd.), and 30 μL of 20 v/v % Sepharose beads (manufactured by GE Healthcare) coated with protein A were added.

Next, K562 cells were solubilized through agitation by aspirating and dispensing using a micropipette in a solubilizing agent (composition: 0.1 w/v % surfactant NP-40 (polyoxy ethylene(9)octylphenyl ether), lx concentration protease inhibitor (product name: Complete manufactured by Roche AG), 50 mM sodium fluoride, 1 mM sodium orthovanadate, and 50 mM tris hydrochloride (pH7.4)) to obtain a cell homogenate. The obtained cell homogenate was centrifuged for 5 minutes at 18000×g, and a supernatant was recovered therefrom to obtain a 10.2 mg/mL K562 solubilized sample. The K562 solubilized sample was diluted by 40-fold, 160-fold, 640-fold, or 2560-fold (the concentration of the solubilized sample after dilution was respectively 2.5%, 4.14%, 1.03%, or 0.04%) using the solubilizing agent. 30 μL each of the obtained dilutions was added to a well. Then, a reaction was caused to occur between CDK1 and the anti-CDK1 antibody or CDK2 and the anti-CDK2 antibody by incubating the 96-well filter plate having added thereto each of the dilutions for 2 hours at 4° C. with shaking.

After the end of the reaction, the beads were recovered from the reaction solution in each well. In the following, for convenience, beads recovered from a reaction solution of the 40-fold dilution are referred to as “Sepharose beads A,” beads recovered from a reaction solution of the 160-fold dilution are referred to as “Sepharose beads B,” beads recovered from a reaction solution of the 640-fold dilution are referred to as “Sepharose beads C,” and beads recovered from a reaction solution of the 2560-fold dilution are referred to as “Sepharose beads D.” The Sepharose beads A to D were each cleaned twice using a beads-cleaning liquid A (composition: 1 w/v % NP-40 and 50 mM tris hydrochloride (pH7.4)).

Next, the Sepharose beads A to D after the cleaning were each cleaned once in a beads-cleaning liquid B (composition: 300 mM sodium chloride and 50 mM tris hydrochloride (pH7.4)). Then, the Sepharose beads A to D were each cleaned once in a beads-cleaning liquid C (composition: 50 mM tris hydrochloride (pH7.4)).

Next, with respect to each of the Sepharose beads A to D that had been cleaned, 50 μL of a CDK substrate solution (composition: 100 ng/μL biotinylated CDK2 substrate peptide (product name: CDK2 substrate (biotinylated) manufactured by Enzo Inc.), the compound 2 represented by formula (x2), 54 mM tris hydrochloride (pH7.4), and 20 mM magnesium chloride) was added to obtain mixtures A to D. The concentration of the compound 2 was a concentration that provided an absorbance of 2 at 362 nm. This absorbance was measured in a manner similar to that in Example 1 (2). The structure of the biotinylated CDK2 substrate peptide was as described next: Biotin-Ahx-His-His-Ala-Ser-Pro-Arg-Lys (SEQ ID NO: 2). It should be noted that “Biotin” represents biotin, and “Ahx” represents aminohexanoic acid (aminocaproic acid).

Each of the obtained mixtures A to D was incubated for 20 minutes at 37° C. with shaking to perform the transphosphorylation by the kinase. By performing this reaction, a DNP group was introduced in the biotinylated substrate peptide. After the end of the phosphorylation, the reaction solutions were each centrifuged for 5 minutes at 760×g, and filtrates were recovered therefrom.

### (3) Detection of Kinase Activity Using Chemical Luminescence

30 μL of a 0.5 v/v % streptavidin labeled magnetic bead-containing HEPES buffer was added to 50 μL of each of the filtrates obtained in (2) described above. By incubating each of the obtained mixtures for 10 minutes at 37° C. with shaking, the biotinylated CDK substrate peptide was captured on the magnetic beads. Then, the magnetic beads that had captured the biotinylated CDK substrate peptide were collected from each of the filtrates using a magnet, and a supernatant was removed therefrom. In the following, magnetic beads obtained from the reaction solution of the mixture A are referred to as “magnetic beads A,” magnetic beads obtained from the reaction solution of the mixture B are referred to as “magnetic beads B,” magnetic beads obtained from the reaction solution of the mixture C are referred to as “magnetic beads C,” and magnetic beads obtained from the reaction solution of the mixture D are referred to as “magnetic beads D.”

The obtained magnetic beads A to D were each cleaned three times using a magnetic beads-cleaning liquid (composition: 0.1 w/v % Tween 20, 20 mM tris hydrochloride (pH7.4), and 138 mM sodium chloride). Next, after the cleaning, 100 μL of a solution containing alkaline phosphatase (ALP) labeled anti-DNP antibody (amount of antibody: 1 unit of ALP activity) was added to each of the magnetic beads A to D. Each obtained mixture was incubated for 20 minutes at 37° C. with shaking for causing a reaction to occur between DNP and the ALP labeled anti-DNP antibody. As the ALP labeled anti-DNP antibody, a labeled antibody obtained by binding ALP manufactured by Oriental Yeast Co., ltd., to a mouse-derived anti-DNP antibody manufactured by Oriental Yeast Co., ltd., via an amino group was used. Next, the magnetic beads A to D after the reaction were collected using a magnet, and supernatants were removed therefrom.

The obtained magnetic beads A to D were cleaned for three times using the magnetic beads-cleaning liquid. Next, 150 μL of a substrate solution containing a chemical luminescence substrate disodium 2-chloro-5-(methoxyspiro {1,2-dioxetane-3,2′-(5′-chloro)tricyclo[3.3.1.1^{3,7}]decan}-4-yl)phenyl phosphate (product name: CDP-star manufactured by Applied Biosystems Corp.) was added to the cleaned magnetic beads A to D. By incubating the obtained mixtures for 5 minutes at 37° C. with shaking, a phosphate ester hydrolysis reaction by ALP was performed.

After the end of the reaction, the obtained reaction solutions were transferred to a black 96-well plate. Then, the black 96-well plate containing the reaction solutions was placed in a luminometer (manufactured by BMG LABTECH Ltd.) to measure the luminescence intensity of each of the reaction solutions. The luminescence intensity obtained when the anti-CDK1 antibody is used is referred to as “luminescence intensity A1,” and the luminescence intensity obtained when the anti-CDK2 antibody is used is referred to as “luminescence intensity A2.”

### (4) Measurement of Background Luminescence Intensity

The luminescence intensity of each of the reaction solutions was measured by performing an operation similar to those in (2) and (3) described above, except for using a rabbit immunoglobulin G (IgG) (manufactured by Calbiochem Corp.) as a control instead of the anti-CDK1 antibody or the anti-CDK2 antibody in (2) and (3) described above. The luminescence intensity obtained when the rabbit IgG is used is referred to as “luminescence intensity B.”

### (5) Examination of Quantifiability of Determining Method According to Present Embodiment

The luminescence intensity A1, the luminescence intensity A2, and the luminescence intensity B were used to obtain a specific luminescence intensity C1 based on CDK1 activity and a specific luminescence intensity C2 based on CDK2 activity in accordance with formula (A) or (B):

Specific Luminescence Intensity C1 based on CDK1 Activity=Luminescence Intensity A1−Luminescence Intensity B (A)

Specific Luminescence Intensity C2 based on CDK2 Activity=Luminescence Intensity A2−Luminescence Intensity B (B).

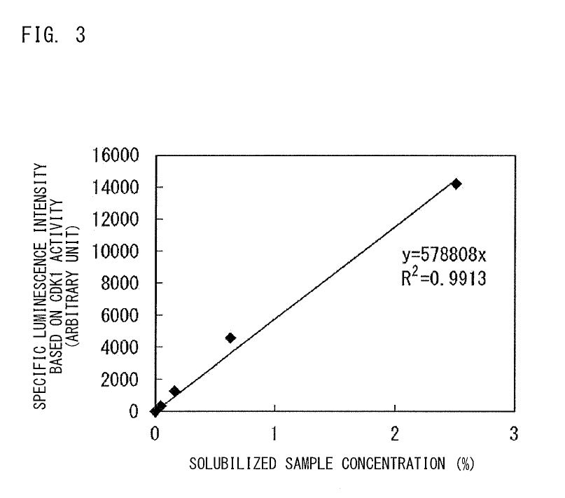

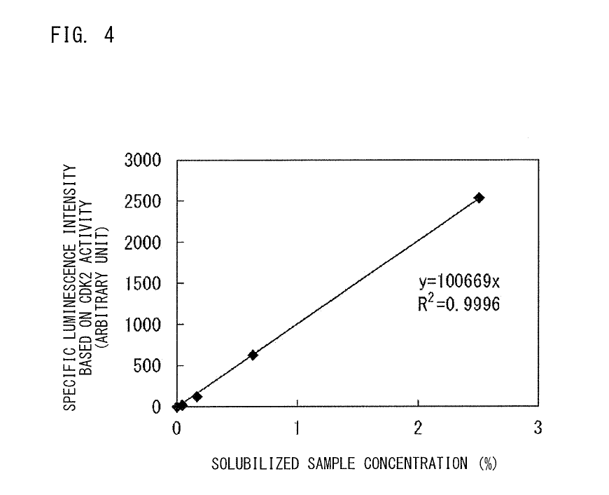

The quantifiability of the determining method according to the present embodiment was evaluated by investigating the relationship between the concentration (solubilized sample concentration) of CDK1 or CDK2 in each of the mixtures A to D, and the specific luminescence intensity C1 or the specific luminescence intensity C2 of each of the reaction solutions obtained using the mixtures A to D. FIG. 3 shows the result of investigating the relationship between the solubilized sample concentration (CDK1 concentration) and the specific luminescence intensity based on CDK1 activity in Example 1. FIG. 4 shows the result of investigating the relationship between the solubilized sample concentration (CDK2 concentration) and the specific luminescence intensity based on CDK2 activity in Example 1.

From the result shown in FIG. 3, it can be understood that the specific luminescence intensity based on CDK1 activity increases in association with an increase in the CDK1 concentration (increase in the solubilized sample concentration). In addition, from the result shown in FIG. 4, it can be understood that the specific luminescence intensity based on CDK2 activity increases in association with an increase in the CDK2 concentration (increase in the solubilized sample concentration). From these results, it can be understood that, with the determining method according to the present embodiment, the activities of kinases such as CDK1 and CDK2 can be determined quantitatively.

### Example 2

### (1) Synthesis of ATPγDNP

The compound 2 represented by formula (x2) was obtained using a technique similar to that in Example 1.

### (2) Kinase Reaction (Transphosphorylation)

In a well of a 96-well filter plate (hydrophilic PVDF membrane manufactured by Millipore Corp.), 70 μL of an immunoprecipitation buffer (a buffer containing 0.1 mass % Nonidet NP-40 and 50 mM tris hydrochloride (pH7.4)) was added. Then, with respect to the immunoprecipitation buffer in the well, 20 μL of an antibody solution containing 16 μg of an anti-CDK1 antibody (manufactured by Operon Co., ltd.), and 30 μL of 20 v/v % Sepharose beads (manufactured by GE Healthcare) coated with protein A were added.

Next, K562 cells were solubilized through agitation by aspirating and dispensing using a micropipette in a solubilizing agent (composition: 0.1 w/v % surfactant NP-40 (polyoxy ethylene(9)octylphenyl ether), lx concentration protease inhibitor (product name: Complete manufactured by Roche AG), 50 mM sodium fluoride, 1 mM sodium orthovanadate, and 50 mM tris hydrochloride (pH7.4)) to obtain a cell homogenate. The obtained cell homogenate was centrifuged for 5 minutes at 18000×g, and a supernatant was recovered therefrom to obtain a 7.58 mg/mL K562 solubilized sample. The K562 solubilized sample was diluted by 86-fold using the solubilizing agent. 30 μL of the obtained dilution was added to a well. Then, a reaction was caused to occur between CDK1 and the anti-CDK1 antibody by incubating the 96-well filter plate having added thereto the dilution for 2 hours at 4° C. with shaking.

After the end of the reaction, the beads were recovered from the obtained reaction solution. The recovered beads were cleaned twice using the beads-cleaning liquid A (composition: 1 w/v % NP-40 and 50 mM tris hydrochloride (pH7.4)). Next, beads after the cleaning were cleaned once in the beads-cleaning liquid B (composition: 300 mM sodium chloride and 50 mM tris hydrochloride (pH7.4)). Furthermore, the beads that had been cleaned were cleaned once using the beads-cleaning liquid C (composition: 50 mM tris hydrochloride (pH7.4)).

Next, with respect to the beads that had been cleaned, 50 μL of the CDK substrate solution (composition: 100 ng/μL biotinylated CDK2 substrate peptide (manufactured by Enzo Inc.), the compound 2 represented by formula (x2), 54 mM tris hydrochloride (pH7.4), and 20 mM magnesium chloride) was added to obtain a mixture. The concentration of the compound 2 was a concentration that provided an absorbance of 2 at 362 nm. This absorbance was measured in a manner similar to that in Example 1 (2).

The obtained mixture was incubated for 20 minutes at 37° C. with shaking to perform the transphosphorylation by the kinase. By performing this reaction, a DNP group was introduced in the biotinylated substrate peptide. After the end of the phosphorylation, the obtained reaction solution was centrifuged for 5 minutes at 760×g (2000 rpm), and a filtrate was recovered therefrom.

### (3) Determination of Kinase Activity Using Chemical Luminescence

30 μL of a 0.5 v/v % streptavidin labeled magnetic bead-containing HEPES buffer was added to 10 μL of the filtrate obtained in (2) described above. By incubating the obtained mixture for 10 minutes at 37° C. with shaking, the biotinylated CDK2 substrate peptide was captured on the magnetic beads. Then, the magnetic beads that had captured the biotinylated CDK2 substrate peptide were collected from the filtrate using a magnet, and a supernatant was removed therefrom.

The obtained magnetic beads were cleaned for three times using the magnetic beads-cleaning liquid (composition: 0.1 w/v % Tween 20, 20 mM tris hydrochloride (pH7.4), and 138 mM sodium chloride). Next, 100 μL of a solution containing the anti-DNP antibody (mouse-derived anti-DNP antibody manufactured by Oriental Yeast Co., ltd.) (amount of antibody: 0.1 ng/μL) was added to the magnetic beads that had been cleaned. The obtained mixture was incubated for 20 minutes at 37° C. with shaking for causing a reaction to occur between the DNP and the anti-DNP antibody. Next, the magnetic beads after the reaction were collected using a magnet, and a supernatant was removed therefrom.

The obtained magnetic beads were cleaned for three times using the magnetic beads-cleaning liquid. Next, 100 μL of a solution containing horseradish peroxidase (hereinafter, also referred to as “HRP”) labeled anti-mouse IgG antibody (manufactured by MBL Co., Ltd.) (amount of antibody: 1000-fold dilution) was added to the magnetic beads that had been cleaned. The obtained mixture was incubated for 60 minutes at 37° C. with shaking for causing a reaction to occur between the IgG of the anti-DNP antibody and the HRP labeled anti-mouse IgG antibody. The magnetic beads after the reaction were collected using a magnet, and a supernatant was removed therefrom.

The obtained magnetic beads were cleaned for three times using the magnetic beads-cleaning liquid. Next, 120 μL of a substrate solution containing a chemical luminescence substrate (product name: Super Signal ELISA Femto manufactured by Pierce Inc.) was added to the magnetic beads that had been cleaned. The obtained mixture was incubated for 5 minutes at 37° C. with shaking.

After the end of the incubation, the obtained reaction solution was transferred to a black 96-well plate. Then, the black 96-well plate containing the reaction solution was placed in a luminometer (manufactured by BMG LABTECH Ltd.) to measure the luminescence intensity A1 of the reaction solution.

### (4) Measurement of Background Luminescence Intensity

The luminescence intensity B of the reaction solution was measured by performing an operation similar to those in (2) and (3) described above, except for using the rabbit immunoglobulin G (IgG) (manufactured by Calbiochem Corp.) as a control instead of the anti-CDK1 antibody in (2) and (3) described above.

### Comparative Example 1

### (1) Kinase Reaction (Transphosphorylation)

In a well of a 96-well filter plate (hydrophilic PVDF membrane manufactured by Millipore Corp.), 70 μL of an immunoprecipitation buffer (a buffer containing 0.1 mass % Nonidet NP-40 and 50 mM tris hydrochloride (pH7.4)) was added. Then, with respect to the immunoprecipitation buffer in the well, 20 μL of an antibody solution containing 16 μg of an anti-CDK1 antibody (manufactured by Operon Co., ltd.), and 30 μL of 20 v/v % Sepharose beads (manufactured by GE Healthcare) coated with protein A were added.

Next, K562 cells were solubilized through agitation by aspirating and dispensing using a micropipette in a solubilizing agent (composition: 0.1 w/v % surfactant NP-40 (polyoxy ethylene(9)octylphenyl ether), lx concentration protease inhibitor (product name: Complete manufactured by Roche AG), 50 mM sodium fluoride, 1 mM sodium orthovanadate, and 50 mM tris hydrochloride (pH7.4)) to obtain a cell homogenate. The obtained cell homogenate was centrifuged for 5 minutes at 18,000×g, and a supernatant was recovered therefrom to obtain a 10.2 mg/mL K562 solubilized sample. The K562 solubilized sample was diluted by 86-fold using the solubilizing agent. 30 μL of the obtained dilution was added to a well. Then, a reaction was caused to occur between CDK1 and the anti-CDK1 antibody by incubating the 96-well filter plate having added thereto the dilution for 2 hours at 4° C. with shaking.

After the end of the reaction, the beads were recovered from the obtained reaction solution. The recovered beads were cleaned twice using the beads-cleaning liquid A (composition: 1 w/v % NP-40 and 50 mM tris hydrochloride (pH7.4)). Next, beads after the cleaning were cleaned once using the beads-cleaning liquid B (composition: 300 mM sodium chloride and 50 mM tris hydrochloride (pH7.4)). Furthermore, the beads that had been cleaned were cleaned once using the beads-cleaning liquid C (composition: 50 mM tris hydrochloride (pH7.4)).

Next, with respect to the beads that had been cleaned, 50 μL of a CDK substrate solution (composition: 100 ng/μL biotinylated CDK2 substrate peptide (manufactured by Enzo Inc.), 2 mM ATPγS (manufactured by Merck & Co., Inc.) as a phosphate-group donor, 54 mM tris hydrochloride (pH7.4), and 20 mM magnesium chloride) was added to obtain a mixture.

The obtained mixture was incubated for 60 minutes at 37° C. with shaking to perform the transphosphorylation by the kinase. By performing this reaction, a monothiophosphate group was introduced to the biotinylated substrate peptide. After the end of the phosphorylation, the obtained reaction solution was centrifuged for 5 minutes at 760×g (2000 rpm), and a filtrate was recovered therefrom.

### (2) Determination of Kinase Activity Using Chemical Luminescence

With respect to 10 μL of the filtrate obtained in (1) described above, a fluorescent labeling reagent (composition: 0.4 mM 5-iodoacetamide fluorescein (5-IAF) (manufactured by Life Technologies Corp.), 285 mM MOPS-NaOH (pH7.4), 4.8 mM EDTA, and 4.9 v/v % DMSO) was added. The obtained mixture was incubated for 10 minutes at 37° C. with shaking while being shielded from light for causing a reaction to occur between 5-IAF and a monothiophosphate group introduced in the biotinylated CDK2 substrate peptide. With this, the monothiophosphate group introduced in the biotinylated CDK2 substrate peptide was labelled with fluorescein. With respect to the obtained reaction solution, a reaction stop solution (composition: 60 mM N-acetyl-L-cysteine and 2M MOPS-NaOH (pH7.4)) was added to terminate the reaction.

30 μL of a 0.5 v/v % streptavidin labeled magnetic bead-containing HEPES buffer was added to 10 μL of the obtained reaction solution. The obtained mixture was incubated for 10 minutes at 37° C. with shaking to cause the biotinylated CDK2 substrate peptide to be captured on the magnetic beads. Then, from the filtrate, the magnetic beads that had captured the biotinylated CDK2 substrate peptide were collected using a magnet, and a supernatant was removed therefrom.

The obtained magnetic beads were cleaned for three times using the magnetic beads-cleaning liquid (composition: 0.1 w/v % Tween 20, 20 mM tris hydrochloride (pH7.4), and 138 mM sodium chloride). Next, 100 μL of a solution containing an anti-fluorescein antibody (manufactured by Acris Antibodies Inc.) (amount of antibody: 0.4 ng/μL) was added to the magnetic beads that had been cleaned. The obtained mixture was incubated for 60 minutes at 37° C. with shaking for causing a reaction to occur between fluorescein and the anti-fluorescein antibody. Next, the magnetic beads after the reaction were collected using a magnet, and a supernatant was removed therefrom.

The obtained magnetic beads were cleaned for three times using the magnetic beads-cleaning liquid. Next, 120 μL of a substrate solution containing a chemical luminescence substrate (product name: Super Signal ELISA Femto manufactured by Pierce Inc.) was added to the magnetic beads that had been cleaned. The obtained mixture was incubated for 5 minutes at 37° C. with shaking.

After the end of the incubation, the obtained reaction solution was transferred to a black 96-well plate. Then, the black 96-well plate containing the reaction solution was placed in a luminometer (manufactured by BMG LABTECH Ltd.) to measure the luminescence intensity A1 of the reaction solution.

### (3) Measurement of Background Luminescence Intensity

The luminescence intensity B of the reaction solution was measured by performing an operation similar to those in (1) and (2) described above, except for using the rabbit immunoglobulin G (IgG) (manufactured by Calbiochem Corp.) as a control instead of the anti-CDK1 antibody in (1) and (2) described above.

Evaluation of Each of the Determining Methods of Example 2 and Comparative Example 1

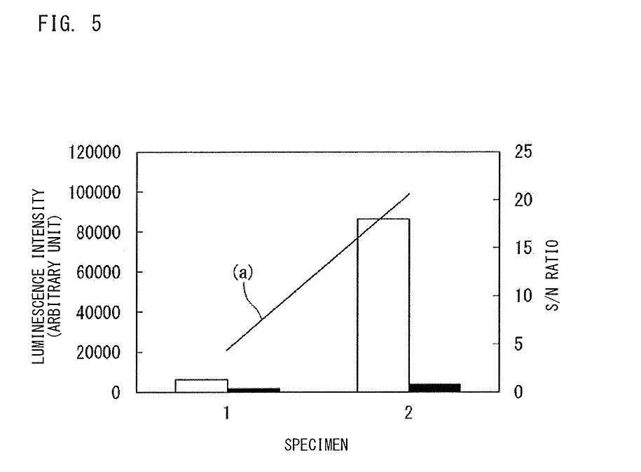

By using the luminescence intensities A1 and B obtained in Example 2, an S/N ratio (luminescence intensity A1/luminescence intensity B) was obtained. Similarly, by using the luminescence intensities A1 and B obtained in Comparative Example 1, an S/N ratio (luminescence intensity A1/luminescence intensity B) was obtained. FIG. 5 shows a result of an evaluation of each of the determining methods of Example 2 and Comparative Example 1. In the figure, line graph (a) shows the S/N ratio of each of the determining methods of Example 2 and Comparative Example 1. Lane 1 shows an evaluation result of the determining method of Comparative Example 1, and lane 2 shows an evaluation result of the determining method of Example 2. A white bar indicates the luminescence intensity A1 based on CDK1 activity, and a black bar indicates the luminescence intensity B of background.

From the results shown in FIG. 5, it can be understood that the S/N ratio of the determining method of Example 2 is significantly higher when compared to the S/N ratio of the determining method of Comparative Example 1. Thus, from these results, it can be understood that, with the determining method according to the present embodiment, kinase activity can be determined with high sensitivity.

### Example 3

100 mg of SM(PEG)₂ was dissolved in 1 mL of DMF, and 0.11 mL of the obtained solution was mixed with a 50 v/v % DMF solution containing 1.5 equivalent of DNP-Lys to obtain a 0.49 mL mixture. The obtained 0.49 mL mixture was diluted by 3.9-fold using 0.1M sodium phosphate (pH7.0). The obtained dilution was left still for 1 hour at 30° C. for causing a reaction to occur between SM(PEG)₂ and DNP-Lys. As a result, 1.88 mL of a solution containing a production intermediate (compound 3) represented by formula (yl) was obtained.

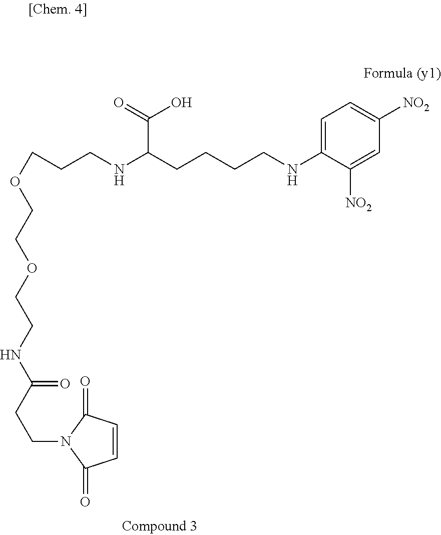 [SMILES: O=C(CCN1C(=O)C=CC1=O)NCCOCCOCCCNC(CCCCNc1ccc([N+](=O)[O-])cc1[N+](=O)[O-])C(=O)O]

ATPγS was dissolved in 88 μL of ultrapure water by a mole number equal to that of SM(PEG)₂ used for the reaction with the DNP-Lys, and 1.81 mL of the solution containing the production intermediate (compound 3) was added thereto. Then, the obtained mixture was left still for 1 hour at 30° C. for causing a reaction to occur between the production intermediate (compound 3) and ATPγS. With respect to 1.9 mL of the obtained solution, a 1 M mercaptoethylamine solution by an amount 1/20 of the volume of the solution was added, and the obtained mixture was left still for 5 minutes at 30° C. to terminate the reaction.

The solution containing the obtained reaction product was purified through reversed-phase chromatography. The purification conditions of the reversed-phase chromatography were those described in the following.

### <Purification Condition>

Detection Wavelength: 260 nm and 360 nm

Used column: C18 reversed-phase column (product name: Redisep Rf C18 manufactured by Teledyne Isco Inc.)

Column temperature: Room temperature

Mobile phase A: 50 mM tetraethylammonium bromide-containing aqueous solution

Mobile phase B: 50 mM tetraethylammonium bromide-containing acetonitrile solution

A concentration gradient of 0 v/v % to 40 v/v % in terms of acetonitrile concentration being used in mobile phases A and B

Flow rate: 5 mL/min

The obtained purified product was concentrated through centrifugation to obtain a compound 4 represented by formula (y2):

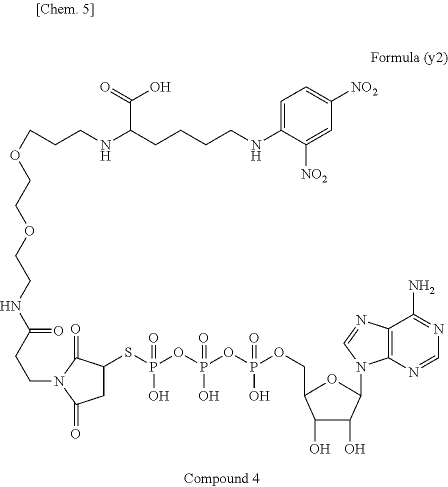 [SMILES: Nc1ncnc2c1ncn2C1OC(COP(=O)(O)OP(=O)(O)OP(=O)(O)SC2CC(=O)N(CCC(=O)NCCOCCOCCCNC(CCCCNc3ccc([N+](=O)[O-])cc3[N+](=O)[O-])C(=O)O)C2=O)C(O)C1O]

Kinase activity was determined in a manner similar to that in Example 2, except for using the compound 4 represented by formula (y2) instead of using the compound 2 represented by formula (x2) in Example 3. As a result, similar to the S/N ratios obtained from the determining methods of Examples 1 and 2, the S/N ratio obtained when using the compound 4 represented by formula (y2) tended to be high when compared to the S/N ratio of the determining method of Comparative Example 1.

In the SEQUENCE LISTING, SEQ ID NO: 1 shows a sequence of a phosphorylation site of a kinase. Xaa at the first position represents Ser (serine residue) or Thr (threonine residue). Xaa at the third position represents any amino acid residue. Xaa at the fourth position represents Lys (lysine residue) or Arg (arginine residue).

SEQ ID NO: 2 shows a sequence of a biotinylated CDK2 substrate peptide. Xaa at the first position represents a biotinylated histidine residue in which a histidine residue is labeled with biotin via aminohexanoic acid (aminocaproic acid).

What is claimed is:

1. A method for determining kinase activity, the method comprising:
(A) mixing a specimen that comprises a kinase, a substrate of the kinase and an adenosine triphosphate (ATP) derivative that comprises a dinitrophenyl (DNP) group to obtain a mixture that comprises a DNP group-containing substrate;
(B) mixing the mixture obtained at the (A) and an antibody that binds to the DNP group to form a complex that comprises the DNP group-containing substrate and the antibody; and
(C) determining an activity of the kinase by detecting the complex,
wherein the ATP derivative is a compound in which the DNP group is bound to a phosphate group at a gamma position of ATP via a linker.

2. The method of claim 1, wherein the complex is formed on a solid support at the (B).

3. The method of claim 2, wherein the substrate is immobilized on the solid support.

4. The method of claim 2, wherein
the complex is formed in a solution at the (B), and
the method further comprises separating the solution and the solid support on which the complex formed, between the (B) and (C).

5. The method of claim 2, wherein the solid support is magnetic particles.

6. The method of claim 1, wherein the kinase is a cyclin-dependent kinase (CDK).

7. The method of claim 1, wherein the kinase is CDK1 or CDK2.

8. The method of claim 1, wherein the substrate is a substrate peptide comprising an amino acid sequence (SEQ ID NO: 1) represented by formula (a1):
Xaa1-Pro-Xaa2-Xaa3 (a1)
(wherein Xaa1 represents a serine residue or a threonine residue, Xaa2 represents any amino acid residue, and Xaa3 represents a lysine residue or an arginine residue).

9. The method of claim 1, wherein:
the antibody is a labeled antibody comprising a labeling substance; and,
at the (C), the activity is determined by detecting the labeling substance in the complex.

10. The method of claim 9, wherein the labeling substance is a labeling enzyme.

11. The method of claim 10, wherein at the (C) a substrate of the labeling enzyme is reacted with the labeling enzyme, the enzymatic reaction generates a signal, and the activity of the kinase is determined by detecting the signal.

12. The method of claim 1, wherein the ATP derivative is a compound represented by formula (I): 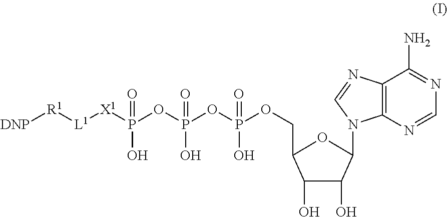
(wherein, X¹ represents a direct binding, an oxygen atom, or a sulfur atom, L¹ represents a linker portion, R¹ represents a reactive group that can be coupled to both the L¹ and the DNP, and DNP represents a dinitrophenyl group).

13. A method for determining kinase activity, the method comprising:
(A) reacting a kinase in a sample, a substrate of the kinase and an adenosine triphosphate (ATP) derivative that comprises a dinitrophenyl (DNP) group to obtain a DNP group-containing substrate;
(B) reacting the DNP group-containing substrate and an antibody that binds to the DNP group to form a complex that comprises the DNP group-containing substrate and the antibody; and
(C) determining an activity of the kinase by detecting the complex,
wherein the ATP derivative is a compound in which the DNP group is bound to a phosphate group at a gamma position of ATP via a linker.

14. The method of claim 13, wherein the substrate is immobilized on a solid support and the complex is formed on a solid support at the (B).

15. The method of claim 14, wherein
the complex is formed in a solution at the (B), and
the method further comprises separating the solution and the solid support on which the complex formed, between the (B) and (C).

16. The method of claim 13, wherein the substrate is a substrate peptide comprising an amino acid sequence (SEQ ID NO: 1) represented by formula (a1):
Xaa1-Pro-Xaa2-Xaa3 (a1)
(wherein Xaa1 represents a serine residue or a threonine residue, Xaa2 represents any amino acid residue, and Xaa3 represents a lysine residue or an arginine residue).

17. The method of claim 1, wherein:
the antibody is a labeled antibody comprising a labeling substance; and,
at the (C), the activity is determined by detecting the labeling substance in the complex.

18. The method of claim 1, wherein the ATP derivative is a compound represented by formula (I): 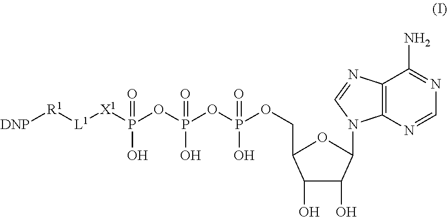
(wherein, X¹ represents a direct binding, an oxygen atom, or a sulfur atom, L¹ represents a linker portion, R¹ represents a reactive group that can be coupled to both the L¹ and the DNP, and DNP represents a dinitrophenyl group).

19. A method for determining kinase activity, the method comprising:
(A) reacting a solid support, a kinase, a substrate of the kinase, and an adenosine triphosphate (ATP) derivative that comprises a dinitrophenyl (DNP) group to obtain a DNP group-containing substrate immobilized on the solid support;
(B) reacting the DNP group-containing substrate and an antibody that binds to the DNP group to form a complex on the solid support, the complex comprising the DNP group-containing substrate and the antibody,
wherein the antibody is labeled with a labeling substance; and
(C) separating the solid support from a liquid phase;
(D) determining an activity of the kinase by detecting the labeling substance of the complex,
wherein the ATP derivative is a compound in which the DNP group is bound to a phosphate group at a gamma position of ATP via a linker.

20. The method of claim 19, wherein the ATP derivative is a compound represented by formula (I): 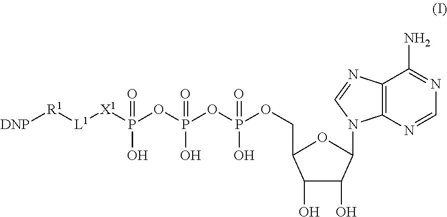
(wherein, X¹ represents a direct binding, an oxygen atom, or a sulfur atom, L¹ represents a linker portion, R¹ represents a reactive group that can be coupled to both the L¹ and the DNP, and DNP represents a dinitrophenyl group).


SEQUENCE LISTING (2 sequences)

SEQ ID NO: 1 - protein, 4 aa, Artificial Sequence
  A sequence of kinase phosphorylation site
XPXX

SEQ ID NO: 2 - protein, 8 aa, Artificial Sequence
  A sequence of biotinylated CDK2 substrate peptide
XHHASPRL